(19) United States
(12) Patent Application Publication
(10) Pub. No.: US 2009/0163743 A1
(43) Pub. Date: Jun. 25, 2009

(54) LUMINESCENCE SYSTEM, METHOD OF LUMINESCENCE, AND CHEMICAL SUBSTANCE FOR LUMINESCENCE

(76) Inventors: Yousuke HOSHI, Tsukuba-shi (JP); Yoshii Morishita, Tsukuba-shi (JP); Satoyuki Nomura, Tsukuba-shi (JP); Yoshihiro Tsuda, Tsukuba-shi (JP); Shigeaki Funyuu, Tsuchiura-shi (JP); Hiroshi Ikeda, Osaka (JP); Hayato Namai, Osaka (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/372,437

(22) Filed: Feb. 17, 2009

Related U.S. Application Data
(62) Division of application No. 10/583,946, filed on Jun. 22, 2006.

(30) Foreign Application Priority Data
Dec. 22, 2003 (JP) P2003-424882

(51) Int. Cl. C07C 43/20; B01J 19/08

(52) U.S. Cl. 568/640; 204/165

(57) ABSTRACT

An object of the present invention is to provide, inexpensively and safely, a luminescence system, a method of luminescence, and a luminescent substance based on a novel luminescence mechanism that luminesces at high efficiency. The present invention relates to a luminescence system, wherein a first chemical substance changes into a second chemical substance having a chemical structure that is different from that of the first chemical substance and thereby luminesces. The present invention preferably relates to the luminescence system, wherein the second chemical substance turns back into the first chemical substance after luminescence.

This application is a Divisional application of prior application Ser. No. 10/583,946, filed Jun. 22, 2006, the contents of which are incorporated herein by reference in their entirety. No. 10/583,946 is a National Stage Application, filed under 35 USC 371, of International Application No. PCT/JP2004/019252, filed Dec. 22, 2004.

### TECHNICAL FIELD

The present invention relates to a luminescence system, a method of luminescence, and a chemical substance for luminescence. The present invention also relates to a luminescent device, and preferably an organic electroluminescent (EL) device, utilizing the luminescence system, the method of luminescence, and the chemical substance for luminescence.

### BACKGROUND ART

Electroluminescent (EL) devices have been attracting attention as, for example, large-area solid state light sources to replace incandescent lamps and gas-filled lamps and, furthermore, they have also been attracting attention as self-luminous displays, and are the most promising alternative to liquid crystal displays (LCDs) in the flat panel display (FPD) field. In particular, an organic electroluminescent (EL) device, in which the device material is formed from an organic material, is being commercialized as a low power consumption full-color flat panel display (FPD).

With regard to the organic electroluminescent (EL) device, both organic low molecular weight type and organic high molecular weight type EL devices have been actively investigated so far, but they have low luminescence efficiency, which gives rise to problems when constructing a full-color display.

As one means for solving this problem, a device utilizing phosphorescence from an excited triplet has been investigated. If phosphorescence from an excited triplet can be utilized, it can be expected that in principle the luminescence quantum yield would be at least three times that obtained when fluorescence from an excited singlet is utilized. Furthermore, while taking into consideration utilization of an exciton resulting from intersystem crossing from the singlet, which has high energy, to the triplet, which has low energy, it can be expected that in principle the luminescence quantum yield would be four times greater than 25%, which is the case when only fluorescence is utilized, that is, it would be 100%.

Examples of research that has been carried out so far into the utilization of luminescence from an excited triplet include publications in which the materials below are used (ref. e.g. M. A. Baldo et al., Appl. Phys. Lett. 1999, 75, 4).
Alq₃: an aluminum-quinolinol complex (tris(8-quinolinolato)aluminum)
α-NPD: N,N′-Di-naphthalen-1-yl-N,N′-diphenyl-biphenyl-4,4′-diamine
CBP: 4,4′-N,N′-dicarbazole-biphenyl
BCP: 2,9-dimethyl-4,7-diphenyl-1,10-phenanthroline
Ir(ppy)₃: iridium-phenylpyridine complex (tris(2-phenylpyridine)iridium)

In addition to the above, there are examples of electroluminescent (EL) devices in which luminescence from an excited triplet is utilized using a metal complex (ref. e.g. Japanese Patent Application Laid-open Nos. 11-329739, 11-256148, and 8-319482).

However, most of the chemical substances that can utilize phosphorescence are metal complexes, and problems with cost, etc. have not been solved. Furthermore, many metal complexes contain a heavy metal. There is therefore a desire for a chemical substance that can utilize phosphorescence even if it does not employ a metal complex.

### DISCLOSURE OF INVENTION

While taking into consideration the above-mentioned conventional problems, it is an object of the present invention to provide, inexpensively and safely, a luminescence system, a method of luminescence, and a luminescent substance based on a luminescence mechanism in which light is emitted in a wide visible light region from short wavelength (blue) to long wavelength (red). It is also an object of the present invention to provide a luminescent device, and preferably an organic electroluminescent (EL) device, utilizing the luminescence system, the method of luminescence, and the luminescent substance.

As a result of an intensive investigation by the present inventors, a luminescence system has been found in which a bond formation or bond cleavage reaction proceeds by injection of an electric charge; after an original chemical substance is changed into a different chemical substance, it luminesces with high efficiency and the original chemical substance is regenerated after the luminescence, and the present invention has thus been accomplished.

That is, the present invention relates to a luminescence system wherein a first chemical substance changes into a second chemical substance having a chemical structure that is different from that of the first chemical substance and thereby luminesces.

Furthermore, the present invention relates to the luminescence system wherein the second chemical substance turns back into the first chemical substance after luminescence.

Moreover, the present invention relates to a method of luminescence of a chemical substance wherein, by injecting an electric charge into a first chemical substance the first chemical substance is formed into an oxidized form or a reduced form of a second chemical substance having a chemical structure that is different from that of the first chemical substance, and by injecting an electric charge that is opposite to the above electric charge an excited state of the second chemical substance is formed, to thereby make it luminesce.

Furthermore, the present invention relates to the method of luminescence wherein the second chemical substance turns back into the first chemical substance after luminescence.

Moreover, the present invention relates to a chemical substance for luminescence wherein a first chemical substance changes into a second chemical substance having a chemical structure that is different from that of the first chemical substance and thereby luminesces.

Furthermore, the present invention relates to the chemical substance for luminescence wherein the second chemical substance turns back into the first chemical substance after luminescence.

Moreover, the present invention relates to the chemical substance for luminescence wherein the second chemical substance is formed via a bond formation reaction from the first chemical substance.

Furthermore, the present invention relates to the chemical substance for luminescence wherein the second chemical substance is formed via a bond cleavage reaction from the first chemical substance.

Moreover, the present invention relates to the chemical substance for luminescence wherein the second chemical substance turns back into the first chemical substance via a bond cleavage reaction.

Furthermore, the present invention relates to the chemical substance for luminescence wherein the second chemical substance turns back into the first chemical substance via a bond formation reaction.

Moreover, the present invention relates to the chemical substance for luminescence wherein the second chemical substance is a n open-shell species having a monoradical or a biradical.

Furthermore, the present invention relates to the chemical substance for luminescence wherein the ground-state multiplicity of the second chemical substance is a triplet.

Moreover, the present invention relates to the chemical substance for luminescence wherein it is represented by Formula (1) below.

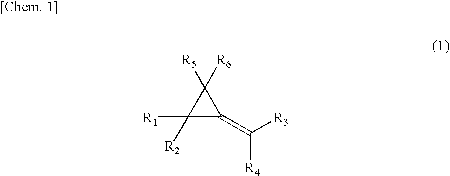

(In the formula, R₁ to R₆ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₁ to R₆ may be identical to or different from each other. Furthermore, R₁ to R₆ may have a substituent selected from the group consisting of —R₇, —OR₈, —SR₉, —OCOR₁₀, —COOR₁₁, —SiR₁₂R₁₃R₁₄, and —NR₁₅R₁₆ (here, R₇ to R₁₆ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₇ to R₁₆ may be identical to or different from each other).)

Furthermore, the present invention relates to the chemical substance for luminescence wherein it is represented by Formula (4) below.

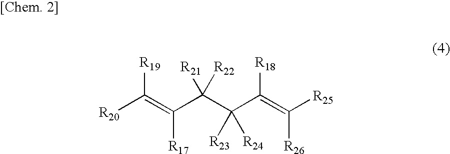

(In the formula, R₁₇ to R₂₆ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₁₇ to R₂₆ may be identical to or different from each other. Furthermore, R₁₇ to R₂₆ may have a substituent selected from the group consisting of —R₂₇, —OR₂₈, —SR₂₉, —OCOR₃₀, —COOR₃₁, —SiR₃₂R₃₃R₃₄, and —NR₃₅R₃₆ (here, R₂₇ to R₃₆ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₂₇ to R₃₆ may be identical to or different from each other).)

Moreover, the present invention relates to the chemical substance for luminescence wherein it is represented by Formula (7) below.

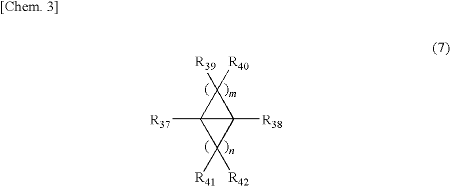

(In the formula, R₃₇ to R₄₂ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₃₇ to R₄₂ may be identical to or different from each other. Furthermore, R₃₇ to R₄₂ may have a substituent selected from the group consisting of —R₄₃, —OR₄₄, —SR₄₅, —OCOR₄₆, —COOR₄₇, —SiR₄₈R₄₉R₅₀, and —NR₅₁R₅₂ (here, R₄₃ to R₅₂ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₄₃ to R₅₂ may be identical to or different from each other). m and n are integers of 1 to 3.)

Furthermore, the present invention relates to the chemical substance for luminescence wherein it is represented by Formula (10) below.

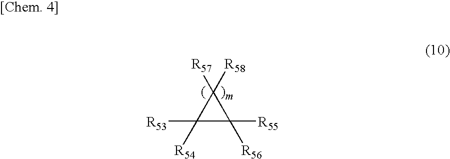

(In the formula, R₅₃ to R₅₈ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₅₃ to R₅₈ may be identical to or different from each other. Furthermore, R₅₃ to R₅₈ may have a substituent selected from the group consisting of —R₅₉, —OR₆₀, —SR₆₁, —OCOR₆₂, —COOR₆₃, —SiR₆₄R₆₅R₆₆, and —NR₆₇R₆₈ (here, R₅₉ to R₆₈ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₅₉ to R₆₈ may be identical to or different from each other). m is an integer of 1 to 3.)

Moreover, the present invention relates to a luminescent device that includes the chemical substance for luminescence.

Furthermore, the present invention relates to an electroluminescent device that includes the chemical substance for luminescence.

Moreover, the present invention relates to a mixture for luminescence that includes the chemical substance for luminescence, and a low molecular weight compound and/or a high molecular weight compound.

The disclosures of the present invention relate to subject matter described in Japanese Patent Application No. 2003-424882 filed on Dec. 22, 2003, and the contents of the disclosures therein are incorporated herein by reference.

### BEST MODE FOR CARRYING OUT THE INVENTION

In organic EL devices up until now, a chemical substance that is responsible for luminescence does not change its chemical structure in an electrically charged state or an excited state, and such a change in the chemical structure has been considered to be undesirable. This is because a different substance is formed as a result of the change in chemical structure, and since the chemical substance that is responsible for luminescence is lost the life span and efficiency of the organic EL device are affected.

On the other hand, as a result of an intensive investigation by the present inventors that was not constrained by such preceding examples, a luminescence system that positively utilizes a change in the chemical structure could be constructed. That is, the luminescence system of the present invention is a luminescence system wherein from a first chemical substance (an original chemical substance) a second chemical substance (a chemical substance having a chemical structure that is different from that of the original chemical substance) is produced and is thereby made to luminesce. In the present invention, the second chemical substance (a chemical substance having a chemical structure that is different from that of the original chemical substance) referred to preferably means a chemical substance obtained as a result of the chemical structure of the first chemical substance (original chemical substance) changing via an intramolecular chemical reaction such as a bond cleavage reaction or a bond formation reaction.

Based on the luminescence system of the present invention, there can be provided a method of luminescence of a chemical substance in which, for example, injecting an electric charge (positive hole or electron) into the first chemical substance induces an intramolecular chemical reaction such as a bond cleavage reaction or a bond formation reaction, thus forming, in an oxidized form or a reduced form, the second chemical substance having a chemical structure that is different from that of the original chemical substance and, furthermore, injecting the opposite electric charge into the oxidized form or reduced form allows an excited state of the second chemical substance to be formed and thereby makes it luminesce.

Furthermore, the chemical substance used in the luminescence system of the present invention is a chemical substance that luminesces after changing into the second chemical substance having a chemical structure that is different from that of the first chemical substance, and is preferably a chemical substance that luminesces after the chemical structure has changed via an intramolecular chemical reaction such as a bond cleavage reaction or a bond formation reaction. Examples of such a chemical substance include a small-membered ring compound such as cyclopropane, methylenecyclopropane, or bicyclopropane and a diolefin such as hexadiene. The small-membered ring compound may be monocyclic or polycyclic.

In the luminescence system of the present invention, the second chemical substance after the change preferably turns back into the first chemical substance rapidly after the luminescence.

Furthermore, the second chemical substance is preferably an open-shell species, and the open-shell species is preferably a monoradical or a biradical.

In the luminescence system of the present invention, the ground-state multiplicity of the second chemical substance is a singlet, a doublet, or a triplet, and in the present invention it is preferable for it to be a triplet in order to obtain a high luminescence quantum yield.

FIGS. 1 and 2 show one embodiment of the luminescence system of the present invention. As shown in FIG. 1, for example, in the case of an organic EL device, after injection of an electric charge from an electrode an original chemical substance (Compound 1) rapidly undergoes a bond cleavage reaction to thus form an oxidized form (Compound 2+) of a chemical substance (a chemical substance having a chemical structure that is different from that of the original chemical substance) that is responsible for luminescence. By injecting the opposite electric charge into the oxidized form, an exciton (Compound 2*) is formed and it luminesces. The chemical substance (Compound 2), which is in the ground state after the luminescence and which has a chemical structure that is different from that of the original chemical structure, rapidly undergoes a bond formation reaction, thus regenerating the original chemical substance (Compound 1).

FIG. 1 shows a process in which a hole is injected, a cation radical is formed, and a bond cleavage reaction proceeds, but the electric charge that is injected and the electric charge of the compound may be different from these. Furthermore, the number of intramolecular chemical reactions until the chemical substance (Compound 2) that is responsible for luminescence is formed from the original chemical substance (Compound 1) is desirably 1 to 10, more desirably 1 to 5, and most desirably 1 to 2. The number of chemical reactions until the original chemical substance is regenerated after the luminescence is desirably 1 to 10, more desirably 1 to 5, and most desirably 1 to 2. When the number of chemical reactions is too many, side reactions easily proceed, and the luminescence efficiency tends to deteriorate.

Furthermore, in the luminescence system of the present invention, as shown in FIG. 2, the sequence of the bond cleavage reaction and the bond formation reaction may be different from that of the case shown in FIG. 1. That is, for example, in the case of an organic EL device, after an electric charge is injected from an electrode, the original chemical substance (Compound 1) rapidly undergoes a bond formation reaction, and an oxidized form (Compound 2+) of the chemical substance (the chemical substance having a chemical structure that is different from that of the original chemical substance) that is responsible for luminescence is thus formed. By injecting the opposite electric charge into this chemical substance, an exciton (Compound 2*) is formed and it luminesces. The chemical substance (Compound 2), which is in the ground state after the luminescence and which has a chemical structure that is different from that of the original chemical structure, rapidly undergoes a bond formation reaction, thus regenerating the original chemical substance.

FIG. 2 shows a process in which a hole is injected, a cation radical is formed, and a bond formation reaction proceeds, but the electric charge that is injected and the electric charge of the compound may be different from these. Furthermore, the number of intramolecular chemical reactions until the chemical substance (Compound 2) that is responsible for luminescence is formed from the original chemical substance (Compound 1) is desirably 1 to 10, more desirably 1 to 5, and most desirably 1 to 2. The number of chemical reactions until the original chemical substance is regenerated after the luminescence is desirably 1 to 10, more desirably 1 to 5, and most desirably 1 to 2. When the number of chemical reactions is too many, side reactions easily proceed, and the luminescence efficiency tends to deteriorate.

The chemical substance of the present invention is now explained by reference to specific compound examples. The compounds shown below can be applied to the above-mentioned luminescence system, method of luminescence, and chemical substance for luminescence, and can preferably be used in a luminescent device, and particularly preferably an organic EL device.

A compound represented by Formula (1) (Compound 1 in FIG. 1) rapidly undergoes a bond cleavage reaction as a result of a hole being injected from an anode, and a compound represented by Formula (2) (Compound 2+ in FIG. 1) is formed. Furthermore, when an electron is injected from a cathode, an excited state compound represented by Formula (3) (Compound 2 in FIG. 1) is formed, and when the compound represented by Formula (3) relaxes to the ground state, it luminesces. The characteristic aspects here are that the ground state of the compound represented by Formula (3) is a triplet, and the 75% of the triplet exciton of the compound represented by Formula (3) formed in the excited state can be utilized efficiently. After the luminescence, the compound represented by Formula (3) rapidly undergoes a bond formation reaction, and the compound represented by Formula (1) is regenerated.

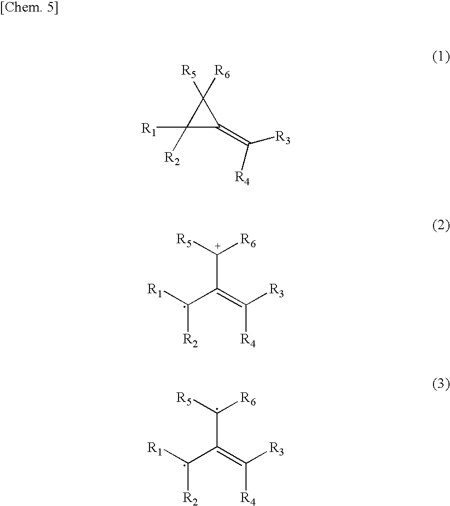

(In the formula, R₁ to R₆ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₁ to R₆ may be identical to or different from each other. Furthermore, R₁ to R₆ may have a substituent selected from the group consisting of —R₇, —OR₈, —SR₉, —OCOR₁₀, —COOR₁₁, —SiR₁₂R₁₃R₁₄, and —NR₁₅R₁₆ (here, R₇ to R₁₆ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₇ to R₁₆ may be identical to or different from each other).)

Examples of the halogen atom include a fluorine atom, a chlorine atom, a bromine atom, and an iodine atom. Examples of the alkyl group include methyl, ethyl, propyl, isopropyl, cyclopropyl, butyl, isobutyl, cyclobutyl, pentyl, isopentyl, neopentyl, cyclopentyl, hexyl, cyclohexyl, heptyl, cycloheptyl, octyl, nonyl, and decyl. Examples of the alkoxy group include methoxy, ethoxy, propoxy, butoxy, tert-butoxy, octyloxy, and tert-octyloxy. Examples of the alkylthio group include methylthio, ethylthio, tert-butylthio, hexylthio, and octylthio. Examples of the aryl group include phenyl, tolyl, xylyl, mesityl, cumenyl, a biphenyl residue, a terphenyl residue, naphthyl, anthryl, and fluorenyl. Examples of the heteroaryl group include a furan residue, a thiophene residue, a pyrrole residue, an oxazole residue, a thiazole residue, an imidazole residue, a pyridine residue, a pyrimidine residue, a pyrazine residue, a triazine residue, a quinoline residue, and a quinoxaline residue. Examples of the aryloxy group include phenoxy, 4-tert-butylphenoxy, 1-naphthyloxy, 2-naphthyloxy, and 9-anthryloxy. Examples of the heteroaryloxy group include pyridinoxy and quinolinoxy. Examples of the arylthio group include phenylthio, 2-methylphenylthio, and 4-tert-butylphenylthio. Examples of the heteroarylthio group include pyridinylthio and quinolinylthio. Examples of the aralkyl group include benzyl, phenethyl, methylbenzyl, and diphenylmethyl.

Examples of —R₇ include a hydrogen atom, a halogen atom such as a fluorine atom, a chlorine atom, a bromine atom, or an iodine atom, a cyano group, a nitro group, methyl, ethyl, propyl, isopropyl, cyclopropyl, butyl, isobutyl, tert-butyl, cyclobutyl, pentyl, isopentyl, neopentyl, cyclopentyl, hexyl, cyclohexyl, heptyl, cycloheptyl, octyl, nonyl, decyl, phenyl, tolyl, xylyl, mesityl, cumenyl, a biphenyl residue, a terphenyl residue, naphthyl, anthryl, fluorenyl, a furan residue, a thiophene residue, a pyrrole residue, an oxazole residue, a thiazole residue, an imidazole residue, a pyridine residue, a pyrimidine residue, a pyrazine residue, a triazine residue, a quinoline residue, a quinoxaline residue, benzyl, phenethyl, methylbenzyl, diphenylmethyl, and halogen-substituted derivatives thereof substituted with a fluorine atom, a chlorine atom, a bromine atom, an iodine atom, etc. Examples of —OR₈ include hydroxyl, methoxy, ethoxy, propoxy, butoxy, tert-butoxy, octyloxy, tert-octyloxy, phenoxy, 4-tert-butylphenoxy, 1-naphthyloxy, 2-naphthyloxy, and 9-anthryloxy. Examples of —SR₉ include mercapto, methylthio, ethylthio, tert-butylthio, hexylthio, octylthio, phenylthio, 2-methylphenylthio, and 4-tert-butylphenylthio. Examples of —OCOR₁₀ include formyloxy, acetoxy, and benzoyloxy. Examples of —COOR₁₁ include carboxyl, methoxycarbonyl, ethoxycarbonyl, tert-butoxycarbonyl, phenoxycarbonyl, and naphthyloxycarbonyl. Examples of —SiR₁₂R₁₃R₁₄ include silyl, trimethylsilyl, triethylsilyl, and triphenylsilyl. Examples of —NR₁₅R₁₆ include amino, N-methylamino, N-ethylamino, N,N-dimethylamino, N,N-diethylamino, N,N-diisopropylamino, N,N-dibutylamino, N-benzylamino, N,N-dibenzylamino, N-phenylamino, and N,N-diphenylamino.

The chemical substance, represented by Formula (3), having a chemical structure that is different from that of the original chemical substance used in the present invention utilizes luminescence due to a transition from an excited triplet to a ground triplet, which is different from phosphorescence emission. Since this transition is spin-allowed, it proceeds more efficiently than phosphorescence emission. In practice, it is possible to obtain a luminescence quantum yield of 1% to a high value of 99% when a compound represented by Formula (3) is used, and this is a material suitable as a luminescent material of an organic EL device.

Furthermore, by changing the substituent denoted by R in Formulae (1) to (3), the luminescence wavelength can be changed within the range from 400 nm to 800 nm, and a substance that luminesces at a given color can be obtained. Specifically, when the conjugation length of the substituent denoted by R in Formulae (1) to (3) is long or the substituent is electron donating, the luminescence wavelength tends to be long. It is preferable for the substituent denoted by R in Formulae (1) to (3) to be a substituent having a conjugated system that can stabilize a cation and a radical.

It is preferable that in Formulae (1) to (3) at least one of R₁ to R₆ is an aryl group. The aryl group may have a substituent denoted by —R₇ or —OR₈. As —R₇, a halogen atom is preferable, and a fluorine atom is more preferable. As —OR₈, an alkoxy group is preferable, and a methoxy group is more preferable.

For example, in Formulae (1) to (3), when R₁ to R₄ are hydrogen atoms and R₅ and R₆ are methoxyphenyl groups, green luminescence can be obtained at a high luminescence quantum yield. Furthermore, in Formulae (1) to (3), when any one of R₁ to R₆ is an aryl group, by introducing a fluoro group into the aryl group the luminescence intensity may be increased, which is preferable. Moreover, in Formulae (1) to (3), when R₁ to R₄ are hydrogen atoms, R₅ is a naphthyl group, and R₆ is a phenyl group, red luminescence can be obtained at a high luminescence quantum yield. This is particularly preferable since red luminescence is difficult to obtain by a conventional metal complex.

The compound represented by Formula (1) may be synthesized by sequentially subjecting an olefin as a starting material to a carbene addition reaction, a methylation reaction, and a base-induced dehydrobromination reaction.

A compound represented by Formula (4) (Compound 1 in FIG. 2) of the present invention rapidly undergoes a bond formation reaction as a result of a hole being injected from the anode, thus forming a compound represented by Formula (5) (Compound 2+ in FIG. 2). Furthermore, when an electron is injected from the cathode, an excited state compound represented by Formula (6) (Compound 2 in FIG. 2) is formed, and luminescence occurs when the compound represented by Formula (6) relaxes to the ground state. After luminescence, the compound represented by Formula (6) rapidly undergoes a bond cleavage reaction, thus regenerating the compound represented by Formula (4).

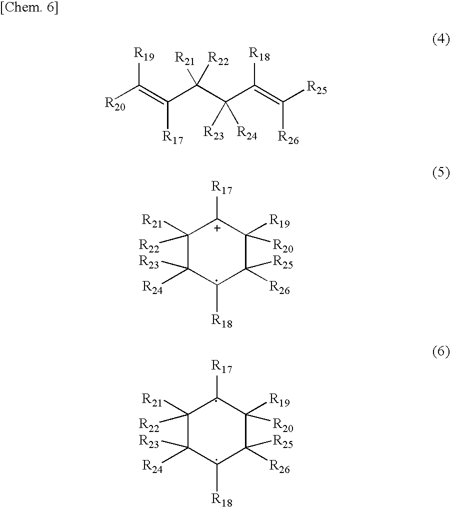

(In the formula, R₁₇ to R₂₆ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₁₇ to R₂₆ may be identical to or different from each other. Furthermore, R₁₇ to R₂₆ may have a substituent selected from the group consisting of —R₂₇, —OR₂₈, —SR₂₉, —OCOR₃₀, —COOR₃₁, —SiR₃₂R₃₃R₃₄, or —NR₃₅R₃₆ (here, R₂₇ to R₃₆ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₂₇ to R₃₆ may be identical to or different from each other).)

Examples of R₁₇ to R₂₆ include the same groups as those cited above for R₁ to R₆, and examples of R₂₇ to R₃₆ include the same groups as those cited above for R₇ to R₁₆.

The substituent denoted by R in Formulae (4) to (6) is preferably a substituent having a conjugated system that can stabilize a cation and a radical.

The compound represented by Formula (4) may be synthesized by a Wittig reaction using a 1,4-diketone.

Furthermore, a compound represented by Formula (7) (Compound 1 in FIG. 1) of the present invention rapidly undergoes a bond cleavage reaction as a result of a hole being injected from the anode, thus forming a compound represented by Formula (8) (Compound 2+ in FIG. 1). Moreover, when an electron is injected from the cathode, an excited state compound represented by Formula (9) (Compound 2 in FIG. 1) is formed, and when the compound represented by Formula (9) relaxes to the ground state, luminescence occurs. After luminescence, the compound represented by Formula (9) rapidly undergoes a bond formation reaction, thus regenerating the compound represented by Formula (7).

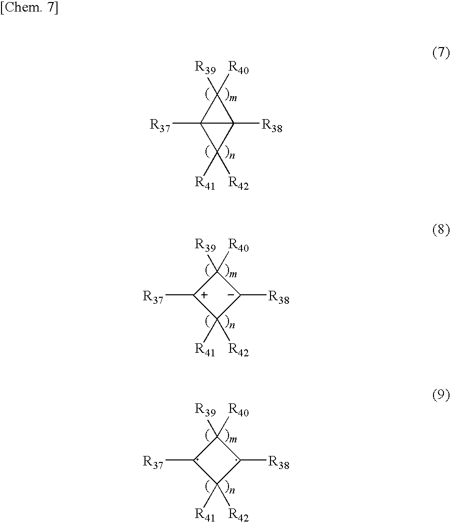

(In the formula, R₃₇ to R₄₂ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₃₇ to R₄₂ may be identical to or different from each other. Furthermore, R₃₇ to R₄₂ may have a substituent selected from the group consisting of —R₄₃, —OR₄₄, —SR₄₅, —OCOR₄₆, —COOR₄₇, —SiR₄₈R₄₉R₅₀, and —NR₅₁R₅₂ (here, R₄₃ to R₅₂ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₄₃ to R₅₂ may be identical to or different from each other). m and n are integers of 1 to 3.)

Examples of R₃₇ to R₄₂ include the same groups as those cited above for R₁ to R₆, and examples of R₄₃ to R₅₂ include the same groups as those cited above for R₇ to R₁₆.

The substituent denoted by R in Formulae (7) to (9) is preferably a substituent having a conjugated system that can stabilize a cation and a radical.

The compound represented by Formula (7) for a case in which m=1 and n=3 may be synthesized by reacting a tosylhydrazone with boron trifluoride so as to make a diazene derivative, followed by denitrogenation by heating. In a case where m=2 and n=2, it may be synthesized by subjecting Formula (4) to a photosensitized electron transfer reaction.

Furthermore, a compound represented by Formula (10) (Compound 1 in FIG. 1) of the present invention rapidly undergoes a bond cleavage reaction as a result of a hole being injected from the anode, thus forming a compound represented by Formula (11) (Compound 2+ in FIG. 1). Furthermore, when an electron is injected from the cathode, an excited state compound represented by Formula (12) (Compound 2 in FIG. 1) is formed, and when the compound represented by Formula (12) relaxes to the ground state, it luminesces. After luminescence, the compound represented by Formula (12) rapidly undergoes a bond formation reaction, thus regenerating the compound represented by Formula (10).

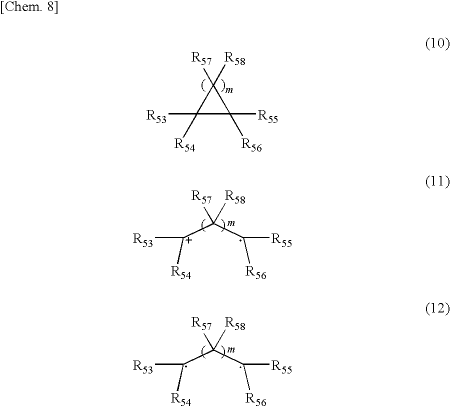

(In the formula, R₅₃ to R₅₈ denote a hydrogen atom, a halogen atom, a cyano group, a nitro group, a hydroxyl group, a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, and R₅₃ to R₅₈ may be identical to or different from each other. Furthermore, R₅₃ to R₅₈ may have a substituent selected from the group consisting of —R₅₉, —OR₆₀, —SR₆₁, —OCOR₆₂, —COOR₆₃, —SiR₆₄R₆₅R₆₆, and —NR₆₇R₆₈ (here, R₅₉ to R₆₈ denote a hydrogen atom, a halogen atom, a cyano group, a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₅₉ to R₆₈ may be identical to or different from each other). m is an integer of 1 to 3.)

Examples of R₅₃ to R₅₈ include the same groups as those cited above for R₁ to R₆, and examples of R₅₉ to R₆₈ include the same groups as those cited above for R₇ to R₁₆.

The substituent denoted by R in Formulae (10) to (12) is preferably a substituent having a conjugated system that can stabilize a cation and a radical.

The compound represented by Formula (10) for a case in which m=1 may be synthesized by subjecting an olefin to a carbene addition reaction. In a case where m=2 or 3, it may be synthesized by subjecting a 1,4-diketone or a 1,5-diketone to a McMurry reaction so as to make a cyclobutene derivative or a cyclopentene derivative, and then to a hydrogenation reaction.

The luminescence system involving a chemical reaction of the present invention can be provided at low cost since the original chemical substance does not contain a metal atom. Furthermore, in the luminescence system of the present invention, since the original chemical substance and the chemical substance that actually produces luminescence have different chemical structures, the chemical substance that actually produces luminescence exhibits a luminescence wavelength that is greatly different from the absorption wavelength of the original chemical substance. In the luminescence system of the present invention, as a highly transparent material, it is preferable to use a chemical substance whose luminescence wavelength is shifted toward longer wavelengths by a chemical reaction.

The luminescence system involving a chemical reaction of the present invention may be used on its own as a light emitting layer of an electroluminescent device. It may also be used as the light emitting layer of the electroluminescent device in a state in which it is dispersed in a host material. The host material is not particularly limited as long as it has the function of receiving a hole from an anode (anode), the function of receiving an electron from a cathode (cathode), the function of transferring a hole and an electron, and the function of giving a hole and an electron to the luminescence system involving a chemical reaction of the present invention, and it is possible to use, for example, a metal complex or a triphenylamine derivative. In particular, when forming an oxidized form of a chemical substance that luminesces in response to injection of a hole, it is desirable to use as the host material a material having high hole injection efficiency and hole transport ability.

A mixture containing the chemical substance for luminescence of the present invention and a low molecular weight compound and/or a high molecular weight compound is preferably used for production of an organic EL device.

Examples of the mixture containing the chemical substance for luminescence of the present invention and the low molecular weight compound include a composition into which is mixed a metal complex such as Alq₃ or a triphenylamine derivative such as α-NPD.

Examples of the mixture containing the chemical substance for luminescence of the present invention and the high molecular weight compound include a polymer composition in which the above-mentioned compound is mixed with a conjugated or nonconjugated polymer. Examples of the conjugated or nonconjugated polymer used as the polymer composition include a polyphenylene derivative, a polyfluorene derivative, a polyphenylene vinylene derivative, a polythiophene derivative, a polyquinoline derivative, a polytriphenylamine derivative, a polyvinylcarbazole derivative, a polyaniline derivative, a polyimide derivative, a polyamideimide derivative, a polycarbonate derivative, a polyacrylic derivative, and a polystyrene derivative, which may be substituted or unsubstituted. As these conjugated or nonconjugated polymers, a polymer obtained by copolymerizing, as another monomer unit as necessary, an arylene and/or heteroarylene monomer unit such as benzene, biphenyl, terphenyl, naphthalene, anthracene, tetracene, fluorene, phenanthrene, chrysene, pyridine, pyrazine, quinoline, isoquinoline, acridine, phenanthroline, furan, pyrrole, thiophene, oxazole, oxadiazole, thiadiazole, triazole, benzoxazole, benzoxadiazole, benzothiadiazole, benzotriazole, or benzothiophene, which may be substituted or unsubstituted, or a monomer unit having a substituted or unsubstituted triphenylamine skeleton such as triphenylamine, N-(4-butylphenyl)-N-diphenylamine, N,N′-diphenyl-N,N′-bis(3-methylphenyl)-[1,1′-biphenyl]-4,4′-diamine, or N,N′-bis(3-methylphenyl)-N,N′-bis(2-naphthyl)-[1,1′-biphenyl]-4,4′-diamine may be used.

With regard to the mixture of the chemical substance for luminescence of the present invention and the low molecular weight compound, the chemical substance for luminescence of the present invention is preferably 0.1 to 50% as a wt % concentration relative to the low molecular weight compound, more preferably 0.5% to 30%, and most preferably 1% to 10%. When it is mixed with, for example, α-NPD as the low molecular weight compound, it is most preferably used at 2% to 10%.

With regard to the mixture of the chemical substance for luminescence of the present invention and the high molecular weight compound, the chemical substance for luminescence of the present invention is preferably 0.1 to 50% as a wt % concentration relative to the high molecular weight compound, more preferably 0.5% to 30%, and most preferably 2% to 10%. When it is mixed with, for example, a polyvinylcarbazole derivative as the high molecular weight compound, it is most preferably used at 2% to 10%.

With regard to the mixture of the chemical substance for luminescence of the present invention, the low molecular weight compound, and the high molecular weight compound, the chemical substance for luminescence of the present invention is preferably 0.1 to 50% as a wt % concentration relative to the total amount of the low molecular weight compound and the high molecular weight compound, more preferably 0.5% to 30%, and most preferably 2% to 10%. When it is mixed with, for example, a mixture of a polyvinylcarbazole derivative and an oxadiazole derivative, it is most preferably used at 2% to 10%.

Furthermore, in the present invention, it is also possible to use for the production of an organic EL device, etc. a high molecular weight compound in which the chemical substance for luminescence of the present invention is incorporated into a high molecular weight compound such as a conjugated or nonconjugated polymer.

The general structure of a device employing the luminescence system involving a chemical reaction of the present invention, specifically, an electroluminescent device of the present invention formed from a mixture of the chemical substance for luminescence of the present invention and a polymer, is described in U.S. Pat. Nos. 4,539,507 and 5,151,629. Furthermore, a polymer-containing electroluminescent device is described in, for example, International Patent Application WO90/13148 or EP-A-0443861.

These devices normally include an electroluminescent layer (light emitting layer) between a cathode (cathode) and an anode (anode), at least one of which is a transparent electrode. Furthermore, at least one electron injection layer and/or electron transfer layer can be inserted between the electroluminescent layer (light emitting layer) and the cathode, and/or at least one positive hole injection layer and/or positive hole transfer layer can be inserted between the electroluminescent layer (light emitting layer) and the anode. Preferred examples of the material of the cathode include a metal or a metal alloy such as Li, Ca, Mg, Al, In, Cs, Ba, Mg/Ag, LiF, or CsF. As the anode, a metal (e.g. Au) or another material having metallic conductivity such as, for example, an oxide (e.g. ITO: indium oxide/tin oxide) on a transparent substrate (e.g. a glass or a transparent polymer) may be used.

In order to use the chemical substance for luminescence of the present invention as a light emitting layer material of the electroluminescent device, it is possible to laminate a layer on a substrate from a solution of the substance on its own or as a mixture or from the substance in a solid state by a method known to a person skilled in the art, such as a resistive heating vapor deposition method, an electron beam vapor deposition method, a sputtering method, an inkjet method, a cast method, an immersion method, a printing method, or a spin coating method, but the method is not limited to the above. Such a laminating method may usually be carried out at a temperature in the range of −20° C. to +500° C., preferably 10° C. to 200° C., and particularly preferably 15° C. to 100° C. The polymer solution thus layered may normally be dried by drying at normal temperature or by drying while heating using a hot plate, etc.

Examples of a solvent used in the solution include chloroform, methylene chloride, dichloroethane, tetrahydrofuran, toluene, xylene, mesitylene, anisole, acetone, methyl ethyl ketone, ethyl acetate, butyl acetate, and ethyl Cellosolve acetate.

Furthermore, the luminescence system involving a chemical reaction of the present invention may be utilized in a luminescent device employing thermoluminescence. By irradiation with energy rays, the luminescent device employing thermoluminescence forms within a solid an oxidized form or a reduced form of a chemical substance having a chemical structure that is different from that of the original chemical substance; by heating, the solid is melted and forms a bond with an opposite electric charge and is thus made to luminesce.

In the luminescent device employing thermoluminescence, the chemical substance of the present invention may be used in a state in which it is dissolved in various types of solvent. The solvent is not particularly limited as long as it is transparent in the visible region, and 1-chlorobutane, 2-methyltetrahydrofuran, and methylcyclohexane, which are highly transparent in a solid state, are preferably used.

Irradiation with energy rays in order to form an oxidized form or a reduced form of the chemical substance having a chemical structure that is different from that of the original chemical substance may be carried out at a temperature equal to or less than the melting temperature of a solvent. However, in order to suppress side reactions, it is preferably carried out at low temperature of −78° C. or below, more preferably at −100° C. or below, and most preferably at −180° C. or below.

As the energy rays for forming an oxidized form or a reduced form of the chemical substance having a chemical structure that is different from that of the original chemical substance, rays that can ionize the original chemical substance can be used. Examples thereof include ultraviolet rays, vacuum ultraviolet rays, X-rays, an electron beam, and γ-rays, and irradiation with γ-rays is the most preferable.

Furthermore, the luminescence system of the present invention may be used in the above-mentioned organic electroluminescent device, the luminescent device employing thermoluminescence and, moreover, in a detection agent for various diagnostic drugs, various types of luminescent probes, an emergency light source, etc. under conditions in which the luminescence phenomenon is sufficiently detectable. In this case, the luminescent substance of the present invention can be bonded, as necessary, to various types of material to be detected under conditions in which the luminescence phenomenon is not impaired. Examples of the material to be detected include biological materials such as antibodies, antigens, various types of proteins such as in vivo proteins and synthetic proteins, polysaccharides, lipids, nucleic acids such as DNA and RNA, various types of macromolecular materials, and molded products thereof.

It is also possible for it to be applied to a missile therapy treatment for, for example, a cancer. Specifically, a specific antibody for a surface antigen of a cancer cell, etc. is modified by the luminescent substance of the present invention, this is placed in the body and made to bond to a cancer cell by an antigen-antibody reaction, and by irradiating the body from outside with a low level of γ-rays, etc. in this state the luminescent substance is made to luminesce, thereby killing the cancer cell by a thermal effect.

In accordance with use of the luminescence system, the method of luminescence, and the chemical substance for luminescence of the present invention, it is possible to provide various types of luminescent devices that luminesce in a wide visible region from short wavelength (blue) to long wavelength (red). For example, when the luminescence system, the method of luminescence, and the chemical substance for luminescence of the present invention are applied to an organic electroluminescent device, even if a metal complex is not used, it is possible to provide a novel device that luminesces in a wide visible region from short wavelength (blue) to long wavelength (red) at high efficiency (internal quantum efficiency) and high luminance. In particular, when the absorption wavelength of the original chemical substance is shorter than the luminescence wavelength of the chemical substance having a structure that is different from that of the original chemical substance, light is not absorbed by the original chemical substance, and a device having high external quantum efficiency can thus be provided.

The chemical substance for luminescence of the present invention is suitably used as a novel organic electroluminescent material. The chemical substances represented by specific structural formulae in the present invention are inexpensive and safe compounds containing no metal, their internal quantum efficiency is high due to the ground state being a triplet state, and they can be used in various types of luminescent devices including an organic electroluminescent device.

### EXAMPLES

The present invention is explained by Examples below, but it should not be construed as being limited thereto, and when the above-mentioned various types of compounds are used, luminescent devices that luminesce at high efficiency can be provided.

### Synthetic Example 1

### Synthesis of 1,1-bis(4-methoxyphenyl)-2-methylenecyclopropane

1,1-Bis(4-methoxyphenyl)ethylene (4.8 g, 20 mmol), bromoform (50.5 g, 200 mmol), a 50% aqueous solution of sodium hydroxide (16 g, 200 mmol), and benzyltriethylammonium chloride (185 mg, 1 mmol) were placed in an Erlenmeyer flask and vigorously stirred at room temperature for 2 days. 100 mL of water was added thereto, extraction with methylene chloride was carried out, and the solvent was removed by distillation. The crude product was purified by column chromatography to give 1,1-bis(4-methoxyphenyl)-2,2-dibromocyclopropane. Yield 76%. Melting point 173-175° C.

A round-bottomed flask was charged with the 1,1-bis(4-methoxyphenyl)-2,2-dibromocyclopropane thus obtained (6.2 g, 15 mmol), iodomethane (4.4 g, 30 mmol), and 100 mL of dry THF, and flushed with nitrogen. A solution of n-butyllithium (11 mL, 18 mmol) was added dropwise thereto while cooling at −78° C., and stirring was carried out at −78° C. for 6 hours. After returning it to room temperature, it was poured into 100 mL of water, and extraction with methylene chloride was carried out. The solvent was removed by distillation and the crude product was purified by column chromatography to give 1,1-bis(4-methoxyphenyl)-2-bromo-2-methylcyclopropane. Yield 82%. Melting point 97-104° C.

A round-bottomed flask was charged with the 1,1-bis(4-methoxyphenyl)-2-bromo-2-methylcyclopropane thus obtained (4.3 g, 12 mmol) and dry dimethylsulfoxide (100 mL), and flushed with nitrogen. Potassium t-butoxide (1.4 g, 12 mmol) was added thereto, and stirring was carried out at room temperature for 2 hours. It was poured into 100 mL of water, and extraction with methylene chloride was carried out. The solvent was removed by distillation and purification was carried out by column chromatography and recrystallization to give 1,1-bis(4-methoxyphenyl)-2-methylenecyclopropane (Chem. (13)). Yield 95%. Melting point 31-32° C. ¹H NMR (200 MHz, CDCl₃) δ 1.81 (dd, J=2.6, 2.0 Hz, 2H), 3.77 (s, 6H), 5.66 (t, J=2.0 Hz, 1H), 5.77 (d, J=2.6 Hz, 1H), 6.81 (AA′BB′, J=8.0 Hz, 4H), 7.20 (AA′BB′, J=8.0 Hz, 4H).

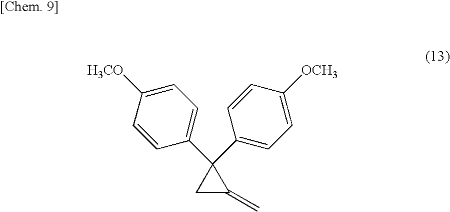

### Synthetic Example 2

### Synthesis of 1-(2-naphthyl)-1-phenyl-2-methylenecyclopropane

A round-bottomed flask was charged with magnesium (1.94 g, 80 mmol) and flushed with nitrogen. A solution of bromobenzene (11 g, 70 mmol) dissolved in 50 mL of dry THF was slowly added dropwise thereto while stirring, thus giving a black Grignard reagent. A solution of 2-acetonaphthone (8.51 g, 50 mmol) dissolved in 50 mL of dry THF was slowly added dropwise thereto and stirred at room temperature for 1 hour. It was further heated and refluxed for 2 hours and then cooled to room temperature, and after water was added thereto extraction with ether was carried out. An oily substance obtained by removing the solvent by distillation was transferred to a round-bottomed flask, 10 mL of THF and 50 mL of a 20% aqueous solution of sulfuric acid were added thereto, and heating and refluxing were carried out for 12 hours. It was cooled to room temperature and neutralized using an aqueous solution of sodium hydroxide. Extraction with ether was carried out and the solvent was removed by distillation. The crude product thus obtained was purified by column chromatography to give 1-(2-naphthyl)-1-phenylethylene. Yield 75%. ¹H-NMR (200 MHz, CDCl₃, δ ppm); 5.56 (s, 1H), 5.60 (s, 1H), 7.33-7.86 (m, 12H).

A round-bottomed flask was charged with 1-(2-naphthyl)-1-phenylethylene (8.64 g, 37.5 mmol), benzyltriethylammonium chloride (10 mg), and bromoform (28.6 g, 113 mmol), and flushed with nitrogen. A 50% aqueous solution of sodium hydroxide (9 mL) was added thereto while stirring, and stirring was carried out at room temperature for 18 hours. After neutralizing with dilute sulfuric acid, extraction with ether was carried out. The solvent was removed by distillation and the crude product thus obtained was formed by column chromatography to give 1-(2-naphthyl)-1-phenyl-2,2-dibromocyclopropane. Yield 44%. ¹H-NMR (200 MHz, CDCl₃, δ ppm); 2.55 (AA′BB′, J=7.8 Hz, 1H), 2.62 (AA′BB′, J=7.8 Hz, 1H), 7.18-7.90 (m, 12H).

A round-bottomed flask was charged with the 1-(2-naphthyl)-1-phenyl-2,2-dibromocyclopropane thus obtained (4.02 g, 10 mmol), iodomethane (2.84 g, 20 mmol), and dry THF (35 mL), and flushed with nitrogen. A solution of n-butyllithium (7.7 mL, 12 mmol) was slowly added thereto while cooling at −78° C., and stirring was carried out for 2 hours. This mixture was returned to room temperature and stirred for a further 1 hour. After water was added thereto, extraction with ether was carried out. The solvent was removed by distillation and the crude product was formed by column chromatography to give 1-(2-naphthyl)-1-phenyl-2-bromo-2-methylcyclopropane. Yield 98%. ¹H-NMR (200 MHz, CDCl₃, δ ppm); 1.78-1.81 (m, 3H), 2.03-2.17 (m, 2H), 7.16-7.91 (m, 12H).

A round-bottomed flask was charged with potassium t-butoxide (1.55 g, 13.8 mmol) and dry dimethylsulfoxide (35 mL) and flushed with nitrogen. A solution of 1-(2-naphthyl)-1-phenyl-2-bromo-2-methylcyclopropane (3.3 g, 9.8 mmol) dissolved in dry dimethylsulfoxide (10 mL) was slowly added dropwise thereto. Stirring was carried out at room temperature for 2 hours, water was added thereto, and extraction with ether was carried out. The solvent was removed by distillation and recrystallization from hexane was carried out to give 1-(2-naphthyl)-1-phenyl-2-methylenecyclopropane (Chem. (14)). Yield 67%. ¹H-NMR (200 MHz, CDCl₃, δ ppm); 1.99 (s, 2H), 5.65 (s, 1H), 5.86 (s, 1H), 7.21-7.80 (m, 12H).

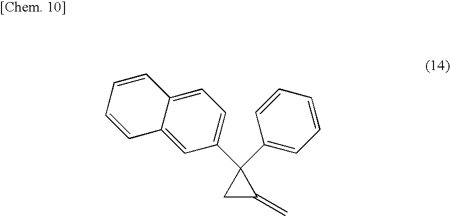

### Synthetic Example 3

### Synthesis of 1-phenyl-2-methylenecyclopropane

Synthesis was carried out in the same manner as in Synthetic Example 1 using styrene as a starting material. ¹H-NMR (200 MHz, CDCl₃, δ ppm); 1.20 (m, 1H), 1.71 (m, 1H), 2.58 (m, 1H), 5.56 (s, 2H), 7.10-7.28 (m, 5H).

### Synthetic Example 4

### Synthesis of 1-methyl-1-phenyl-2-methylenecyclopropane

Synthesis was carried out in the same manner as in Synthetic Example 1 using α-methylstyrene as a starting material. ¹H-NMR (200 MHz, CDCl₃, δ ppm); 1.38-1.40 (m, 2H), 1.53 (s, 1H), 5.47 (s, 1H), 5.58 (s, 1H), 7.11-7.32 (m, 5H).

### Synthetic Example 5

### Synthesis of 1-(1-naphthyl)-1-phenyl-2-methylenecyclopropane

Synthesis was carried out in the same manner as in Synthetic Example 2 using bromobenzene and 1-acetonaphthone as starting materials. ¹H-NMR (200 MHz, CDCl₃, δ ppm); 2.01 (ddd, J=8.8 Hz, J=2.7 Hz, J=2.7 Hz, 1H), 2.14 (ddd, J=8.8 Hz, J=2.7 Hz, J=2.7 Hz, 1H), 5.67 (br, 1H), 5.89 (dd, J=2.7 Hz, J=2.7 Hz, 1H), 7.06-8.13 (m, 12H).

### Synthetic Example 6

### Synthesis of 1-phenyl-1-(4-phenylphenyl)-2-methylenecyclopropane

Synthesis was carried out in the same manner as in Synthetic Example 2 using 4-bromobiphenyl and acetophenone as starting materials. ¹H-NMR (200 MHz, CDCl₃, δ ppm); 1.94 (dd, J=2.4 Hz, J=2.2 Hz, 2H), 5.63 (dd, J=1.8 Hz, J=1.8 Hz, 1H), 5.84 (dd, J=2.6 Hz, J=2.4 Hz, 1H), 7.26-7.59 (m, 14H).

### Synthetic Example 7

### Synthesis of 1-(4-bromophenyl)-1-phenyl-2-methylenecyclopropane

A Wittig reagent was prepared under a nitrogen atmosphere from a solution of potassium t-butoxide (6.06 g, 54 mmol) in dry THF (65 mL) and a methyl phosphonium salt (27.3 g, 68 mmol). A solution of 4-bromobenzophenone (11.8 g, 45 mmol) in dry THF (125 mL) was added dropwise thereto, stirring at room temperature was carried out for 1 hour, extraction with ether was then carried out, and the solvent was removed by distillation. Purification was carried out by column chromatography to give 1-(4-bromophenyl)-1-phenylethylene. Yield 96%.

Synthesis of 1-(4-bromophenyl)-1-phenyl-2-methylenecyclopropane was carried out by the same method as in Synthetic Example 1 using the 1-(4-bromophenyl)-1-phenylethylene thus obtained. ¹H-NMR (200 MHz, CDCl₃, δ ppm); 1.84 (d, J=13.6 Hz, 1H), 1.92 (d, J=13.6 Hz, 1H), 5.6 (s, 1H), 5.78 (s, 1H), 7.13 (d, J=8.6 Hz, 2H), 7.20_{—}7.28 (m, 5H), 7.38 (d, J=8.6 Hz, 2H).

### Synthetic Example 8

### Synthesis of 1,1-bis(4-fluorophenyl)-2-methylenecyclopropane

Synthesis was carried out in the same method as in Synthetic Example 7 using 4,4′-difluorobenzophenone as a starting material. ¹H-NMR (200 MHz, CDCl₃, δ ppm); 1.85 (dd, J=2.6 Hz, J=2.0 Hz, 2H), 5.61 (dd, J=2.1 Hz, J=1.8 Hz, 1H), 5.79 (dd, J=2.6 Hz, J=1.8 Hz, 1H), 6.91-7.00 (m, 4H), 7.19-7.26 (m, 4H).

### Synthetic Example 9

### Synthesis of 1,1-diphenyl-2-methylenecyclopropane

Synthesis was carried out in the same method as in Synthetic Example 7 using benzophenone as a starting material. ¹H-NMR (200 MHz, CDCl₃, δ ppm); 1.90 (dd, J=2.7 Hz, J=2.0 Hz, 2H), 5.60 (t, J=2.0 Hz, 1H), 5.80 (d, J=2.7 Hz, 1H), 7.21-7.30 (m, 10H).

### Synthetic Example 10

### Synthesis of 1-(3,5-dibromophenyl)-1-phenyl-2-methylenecyclopropane

A round-bottomed flask was charged with 1,3,5-tribromobenzene (6.3 g, 20 mmol) and dry ether (150 mL) under a nitrogen atmosphere. A solution of n-butyllithium (12.5 mL, 20 mmol) was added thereto while cooling at −78° C., and stirring at −78° C. was carried out for 2 hours. A solution of N,N-dimethylacetamide (1.92 g, 22 mmol) in dry ether (15 mL) was further added dropwise thereto. The temperature was gradually returned from −78° C. to room temperature, stirring was carried out for 20 hours, extraction with ether was then carried out, and the solvent was removed by distillation. Purification was carried out by column chromatography and recrystallization to give 3,5-dibromoacetophenone. Yield 41%.

A Grignard reagent was prepared from a solution of bromobenzene (4.98 g, 32 mmol) in dry THF (15 mL) and magnesium (717 mg, 30 mmol) under a nitrogen atmosphere. A solution of 3,5-dibromoacetophenone (6.30 g, 23 mmol) in dry THF (30 mL) was added dropwise thereto, stirring was carried out at room temperature for 1 hour, and heating and refluxing were then carried out for 15 hours. After the temperature was returned to room temperature, extraction with ether was carried out, and the solvent was removed by distillation. The residue was transferred to a round-bottomed flask, toluene (100 mL) and p-toluenesulfonic acid monohydrate (432 mg, 2.3 mmol) were added thereto, and heating and refluxing were carried out for 15 hours. After the solvent was removed by distillation, purification was carried out by vacuum distillation to give 1-(3,5-dibromophenyl)-1-phenylethylene. Yield 78%.

By carrying out a reaction using the 1-(3,5-dibromophenyl)-1-phenylethylene thus obtained in the same manner as in Synthetic Example 1,1-(3,5-dibromophenyl)-1-phenyl-2-methylenecyclopropane was synthesized. ¹H-NMR (200 MHz, CDCl₃, δ ppm); 1.84 (d, J=9.2 Hz, 1H), 1.95 (d, J=9.2 Hz, 1H), 5.64 (s, 1H), 5.82 (s, 1H), 7.23-7.31 (m, 7H), 7.49 (s, 1H).

### Synthetic Example 11

### Synthesis of 1-(3,5-diphenylphenyl)-1-phenyl-2-methylenecyclopropane

A round-bottomed flask was charged, under a nitrogen atmosphere, with 1-(3,5-dibromophenyl)-1-phenyl-2-methylenecyclopropane (130 mg, 0.36 mmol), phenylboronic acid (100 mg, 0.82 mmol), palladium tetrakistriphenylphosphine (60.2 mg, 0.054 mmol), potassium carbonate (986 mg, 7.2 mmol), tetrabutylammonium chloride (27.8 mg, 0.089 mmol), benzene (7 mL), and water (7 mL), and stirring was carried out at 75° C. for 48 hours. After the temperature was returned to room temperature, extraction with ether was carried out, and the solvent was removed by distillation. Purification was carried out by column chromatography to give 1-(3,5-diphenylphenyl)-1-phenyl-2-methylenecyclopropane. Yield 93%. ¹H-NMR (200 MHz, CDCl₃, 8 ppm); 1.97 (m, 2H), 5.65 (s, 1H), 5.88 (s, 1H), 7.21-7.65 (m, 18H).

### Synthetic Example 12

### Synthesis of 1,5-di(4-methoxyphenyl)bicyclo[3.1.0]hexane

Under a flow of nitrogen, a round-bottomed flask was charged with triphenylmethylphosphonium iodide (8.08 g, 20 mmol) and dry THF (60 mL), and potassium t-butoxide (2.24 g, 20 mmol) was added thereto and stirred at room temperature for 30 minutes to give a yellow solution. This solution was slowly added to a solution of 1,5-di(4-methoxyphenyl)-1,5-pentadione (6.25 g, 20 mmol) in dry THF (140 mL) placed in another round-bottomed flask. After stirring for 12 hours, water was added thereto, and extraction with ether was carried out. Purification was carried out by column chromatography to give 1,5-di(4-methoxyphenyl)-5-hexen-1-one. Yield 56%. ¹H-NMR (200 MHz, CDCl₃, δ ppm); 1.89 (tt, J=7.3, 7.3 Hz, 2H), 2.59 (t, J=7.3 Hz, 2H), 2.92 (t, J=7.3 Hz, 2H), 3.81 (s, 3H), 3.86 (s, 3H), 5.00 (s, 1H), 5.25 (s, 1H), 6.87 (AA′XX′, J=6.5 Hz, 2H), 6.91 (AA′XX′, J=8.8 Hz, 2H), 7.38 (AA′XX′, J=8.8 Hz, 2H), 7.89 (AA′XX′, J=6.5 Hz, 2H).

Under a flow of nitrogen, a flask was charged with 1,5-di(4-methoxyphenyl)-5-hexen-1-one (1.55 g, 5 mmol) and methanol (15 mL), and a solution of 4-tosylhydrazone (1.02 g, 5.5 mmol) in methanol (5 mL) was added thereto all at once. Stirring was carried out at room temperature for 5 days, and a powder thus precipitated was filtered. This powder was washed well with hexane to give 5-(4-tosylhydrazono)-1,5-(4-methoxyphenyl)-pentan-1-one. Yield 93%. ¹H-NMR (200 MHz, CDCl₃, δ ppm); 1.54 (m, 2H), 2.39 (s, 3H), 2.50 (m, 4H), 3.80 (s, 3H), 3.84 (s, 3H), 4.98 (s, 1H), 5.27 (s, 1H), 6.78 (AA′XX′, J=9.0 Hz, 2H), 6.89 (AA′XX′, J=8.8 Hz, 2H), 7.26 (AA′XX′, J=8.4 Hz, 2H), 7.31 (AA′XX′, J=8.8 Hz, 2H), 7.45 (AA′XX′, J=9.0 Hz, 2H), 7.79 (AA′XX′, J=8.4 Hz, 2H).

Under a flow of nitrogen, a flask was charged with 5-(4-tosylhydrazono)-1,5-(4-methoxyphenyl)-pentan-1-one (476.8 mg, 1.0 mmol) and 10 mL of dry methylene chloride, trifluoroborane eth erate (0.14 mL, 1.1 mmol) was added thereto while shielding it from light in an ice bath, and stirring was carried out for 20 minutes. Stirring was carried out at room temperature for a further 3 hours, and water (5 mL) was added thereto. In a dark place, extraction with methylene chloride was carried out, and the solvent was removed by distillation. Benzene (10 mL) was added thereto, and heating and refluxing were carried out under nitrogen for 2 hours. The solvent was removed by distillation and purification was carried out by column chromatography and recrystallization to give 1,5-di(4-methoxyphenyl)bicyclo[3.1.0]hexane. Yield 40%. Melting point 92-93° C. ¹H-NMR (200 MHz, CDCl₃, δ ppm); 1.24-1.50 (m, 3H), 1.79 (m, 1H), 2.05-2.37 (m, 4H), 3.72 (s, 6H), 6.69 (d, J=8.8 Hz, 4H), 6.98 (d, J=8.8 Hz, 4H).

### Example 1

### Detection of Trimethylenemethane Cation Radical by CIDEP Method

A CIDEP spectrum was measured by a conventional method (ref. e.g. 4th Edition of Jikken Kagaku Koza (Experimental Chemistry), Vol. 8, Spectroscopy III, p. 541, 1992, Maruzen). Transient changes were monitored by a digital oscilloscope using an EX600 excimer laser manufactured by GSI Lumonics Inc. as a light source and an E-109 electron spin resonance measurement apparatus manufactured by Varian Inc. and an ESP-380E electron spin resonance measurement apparatus manufactured by Bruker GmbH. Chloranil (10 mM) was added as a sensitizer to a DMSO solution of 1,1-bis(4-methoxyphenyl)-2-methylenecyclopropane (50 mM) obtained in Synthetic Example 1. When a CIDEP spectrum was measured while applying an XeCl laser (441 nm) employing coumarin 440 to this solution at room temperature, the spectrum shown in FIG. 3 was obtained. It was confirmed from comparison with a reference (H. Ikeda et al., J. Am. Chem. Soc., 2003, 125, 9147-9157) that a trimethylenemethane cation radical was produced.

### Example 2

### Detection of Trimethylenemethane Biradical by ESR

Measurement of an ESR spectrum employed an ESP-380E electron spin resonance measurement apparatus manufactured by Bruker GmbH. Anthraquinone (50 mM) was added as a sensitizer to a methylene chloride solution of 1,1-bis(4-methoxyphenyl)-2-methylenecyclopropane (50 mM) obtained in Synthetic Example 1. When this solution was cooled to 20K and an ESR spectrum was measured by applying a GCR-14 YAG laser (355 nm) manufactured by Quanta-Ray Inc., the spectrum shown in FIG. 4 was obtained. It was confirmed from comparison with a reference (H. Ikeda et al., J. Am. Chem. Soc., 1998, 120, 5832-5833) that a trimethylenemethane cation radical was produced. When it was further cooled to 5K and the change in signal strength due to temperature change was monitored, it was confirmed that this trimethylenemethane biradical was in the ground triplet state.

### Example 3

### Transient Absorption Spectrum of Trimethylenemethane Cation Radical

A transient absorption spectrum was measured by a conventional method (ref. e.g. 4th Edition of Jikken Kagaku Koza (Experimental Chemistry), Vol. 7, Spectroscopy II, p. 275, 1992, Maruzen). An EX600 excimer laser manufactured by GSI Lumonics Inc. was used as a light source, and a spectrum was measured using a USP-600 detector manufactured by Unisoku Co., Ltd. Tetracyanobenzene (0.8 mM) was added as a sensitizer to an acetonitrile solution of 1,1-bis(4-methoxyphenyl)-2-methylenecyclopropane (3 mM) obtained in Synthetic Example 1. When a transient absorption spectrum was measured while applying an XeCl laser (308 nm) to this solution at room temperature, the trimethylenemethane cation radical absorption spectrum shown in FIG. 5 was obtained, and the maximum absorption wavelength was 500 nm.

### Example 4

### Observation of Thermoluminescence

A methylcyclohexane solution of 1,1-bis(4-methoxyphenyl)-2-methylenecyclopropane (5 mM) obtained in Synthetic Example 1 was placed in a synthetic quartz cell, and degassed and sealed. This cell was immersed in liquid nitrogen so as to solidify the solution, and γ-rays from cobalt 60 were applied for 40 hours. When an absorption spectrum was measured in liquid nitrogen with an HP8452A spectrophotometer manufactured by Hewlett-Packard, absorption was observed at 510 nm. From comparison with Example 3, this absorption was identified as being due to a trimethylenemethane cation radical. When this cell was taken out of liquid nitrogen and allowed to warm, a green luminescence was observed. When a luminescence spectrum was measured with a PMA-11 multichannel spectral analyzer manufactured by Hamamatsu Photonics K. K., the luminescence spectrum shown in FIG. 6 was obtained, and the maximum emission wavelength was 561 nm.

That is, the trimethylenemethane cation radical was formed by one-electron oxidation of 1,1-bis(4-methoxyphenyl)-2-methylenecyclopropane, and luminescence from trimethylenemethane biradical proceeded by recombination with an electron.

### Example 5

### Fabrication of Organic EL Device Using 1,1-bis(4-methoxyphenyl)-2-methylenecyclopropane

A mixture of polyvinylcarbazole (77 parts by weight), 2-(4-biphenylyl)-5-(4-t-butylphenyl)-1,3,4-oxadiazole (15 parts by weight), and 1,1-bis(4-methoxyphenyl)-2-methylenecyclopropane (8 parts by weight) obtained in Synthetic Example 1 was dissolved in anisole (concentration 2 wt %) to give a coating solution. A glass substrate patterned with ITO (indium tin oxide) at a width of 1.6 mm was spin-coated under a dry nitrogen atmosphere, thus forming a polymer light emitting layer (thickness 100 nm) in which 1,1-bis(4-methoxyphenyl)-2-methylenecyclopropane was present. Subsequently, under a dry nitrogen atmosphere, this was heated and dried at 80° C./5 minutes on a hot plate. The glass substrate thus obtained was transferred to a vacuum vapor deposition apparatus, and electrodes of Ca (thickness 20 nm) and Al (thickness 100 nm) were formed in sequence on the light emitting layer. The properties of the organic EL device were measured at room temperature using a 4140B picoammeter manufactured by Hewlett-Packard for current-voltage characteristics and using an SR-3 manufactured by Topcon for luminance. When a voltage was applied using the ITO as an anode and the Ca/Al as a cathode, a pale yellow luminescence was observed at about 30 V. The luminescence spectrum is shown by the solid line in FIG. 8.

### Comparative Example 1

An ITO/polymer light emitting layer/Ca/Al device was fabricated in the same manner as in Example 5 except that 1,1-bis(4-methoxyphenyl)-2-methylenecyclopropane was not added. When the ITO/polymer light emitting layer/Ca/Al device thus obtained was connected to a power source, and a voltage was applied using the ITO as an anode and the Ca/Al as a cathode, a blue luminescence was observed at about 20 V. The luminescence spectrum is shown by the broken line in FIG. 8.

### Example 6

### Observation of Thermoluminescence

A methylcyclohexane solution of 1-(2-naphthyl)-1-phenyl-2-methylenecyclopropane (5 mM) obtained in Synthetic Example 2 was placed in a synthetic quartz cell, and degassed and sealed. This cell was immersed in liquid nitrogen so as to solidify the solution, and γ-rays from cobalt 60 were applied for 40 hours. When this cell was taken out of liquid nitrogen and allowed to warm, a red luminescence was observed. The luminescence spectrum is shown in FIG. 9.

### Example 7

### Fabrication of Organic EL Device Using 1-(2-naphthyl)-1-phenyl-2-methylenecyclopropane

A mixture of polyvinylcarbazole (72 parts by weight), 2-(4-biphenylyl)-5-(4-t-butylphenyl)-1,3,4-oxadiazole (21 parts by weight), and 1-(2-naphthyl)-1-phenyl-2-methylenecyclopropane (7 parts by weight) obtained in Synthetic Example 2 was dissolved in anisole (concentration 2 wt %) to give a coating solution. When an organic EL device was fabricated in the same manner as in Example 5, and a voltage was applied using the ITO as an anode and the Ca/Al as a cathode, a pink luminescence was observed at about 20 V. The luminescence spectrum is shown by the solid line in FIG. 10.

### Comparative Example 2

An organic EL device was fabricated in the same manner as in Example 7 except that 1-(2-naphthyl)-1-phenyl-2-methylenecyclopropane was not added. When the organic EL device thus obtained was connected to a power source, and a voltage was applied using the ITO as an anode and the Ca/Al as a cathode, a blue luminescence was observed at about 15 V. The luminescence spectrum is shown by the broken line in FIG. 10.

### Example 8

### Observation of Thermoluminescence

A methylcyclohexane solution of 1,5-di(4-methoxyphenyl)bicyclo[3.1.0]hexane (5 mM) obtained in Synthetic Example 12 was placed in a synthetic quartz cell, and degassed and sealed. This cell was immersed in liquid nitrogen so as to solidify the solution, and γ-rays from cobalt 60 was applied for 40 hours. When this cell was taken out of liquid nitrogen and allowed to warm, a yellow luminescence was observed. The luminescence spectrum is shown in FIG. 11.

### Example 9

### Fabrication of Organic EL Device Using 1,5-di(4-methoxyphenyl)bicyclo[3.1.0]hexane

A mixture of polyvinylcarbazole (72 parts by weight), 2-(4-biphenylyl)-5-(4-t-butylphenyl)-1,3,4-oxadiazole (21 parts by weight), and 1,5-di(4-methoxyphenyl)bicyclo[3.1.0]hexane (7 parts by weight) obtained in Synthetic Example 12 was dissolved in anisole (concentration 2 wt %) to give a coating solution. When an organic EL device was fabricated in the same manner as in Example 5, and a voltage was applied using the ITO as an anode and the Ca/Al as a cathode, a pale pink luminescence was observed at about 25 V. The luminescence spectrum is shown by the solid line in FIG. 12.

### Comparative Example 3

An organic EL device was fabricated in the same manner as in Example 9 except that 1,5-di(4-methoxyphenyl)bicyclo[3.1.0]hexane was not added. When the organic EL device thus obtained was connected to a power source, and a voltage was applied using the ITO as an anode and the Ca/Al as a cathode, a blue luminescence was observed at about 15 V. The luminescence spectrum is shown by the broken line in FIG. 12.

### BRIEF DESCRIPTION OF DRAWINGS

FIG. 1 is a schematic diagram showing one embodiment of the luminescence system of the present invention.

FIG. 2 is a schematic diagram showing another embodiment of the luminescence system of the present invention.

FIG. 3 is a CIDEP spectrum of a trimethylenemethane cation radical observed in Example 1.

FIG. 4 is an ESR spectrum of a trimethylenemethane biradical observed in Example 2.

FIG. 5 is a transient absorption spectrum of a trimethylenemethane cation radical observed in Example 3.

FIG. 6 is a luminescence spectrum of a luminescent device employing thermoluminescence observed in Example 4.

FIG. 7 is a photographic diagram showing luminescence from the luminescent device employing thermoluminescence observed in Example 4.

FIG. 8 shows luminescence spectra of luminescent devices employing electroluminescence observed in Example 5 and Comparative Example 1.

FIG. 9 is a luminescence spectrum of a luminescent device employing thermoluminescence observed in Example 6.

FIG. 10 shows luminescence spectra of luminescent devices employing electroluminescence observed in Example 7 and Comparative Example 2.

FIG. 11 is a luminescence spectrum of a luminescent device employing thermoluminescence observed in Example 8.

FIG. 12 shows luminescence spectra of luminescent devices employing electroluminescence observed in Example 9 and Comparative Example 3.

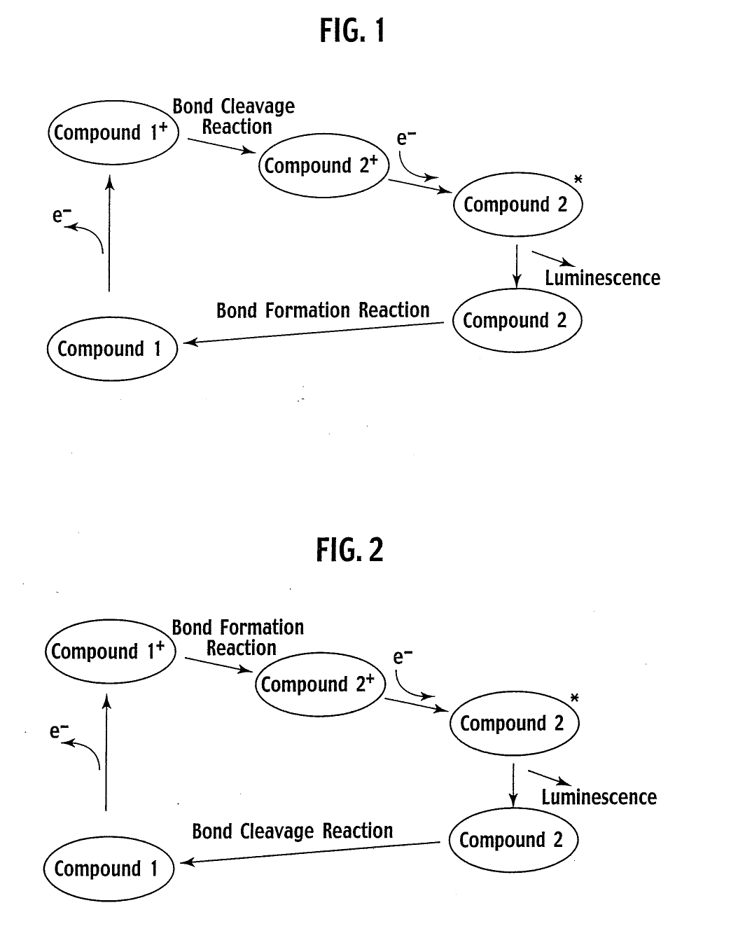

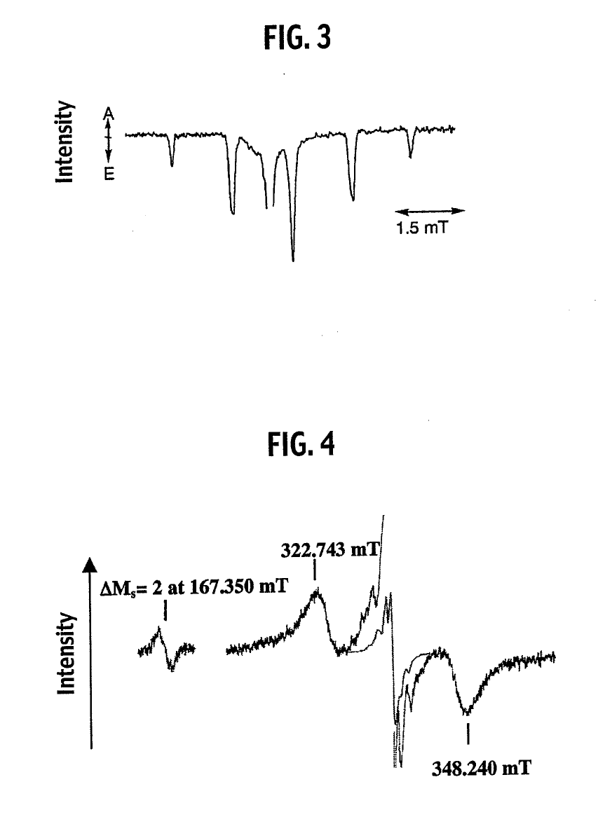

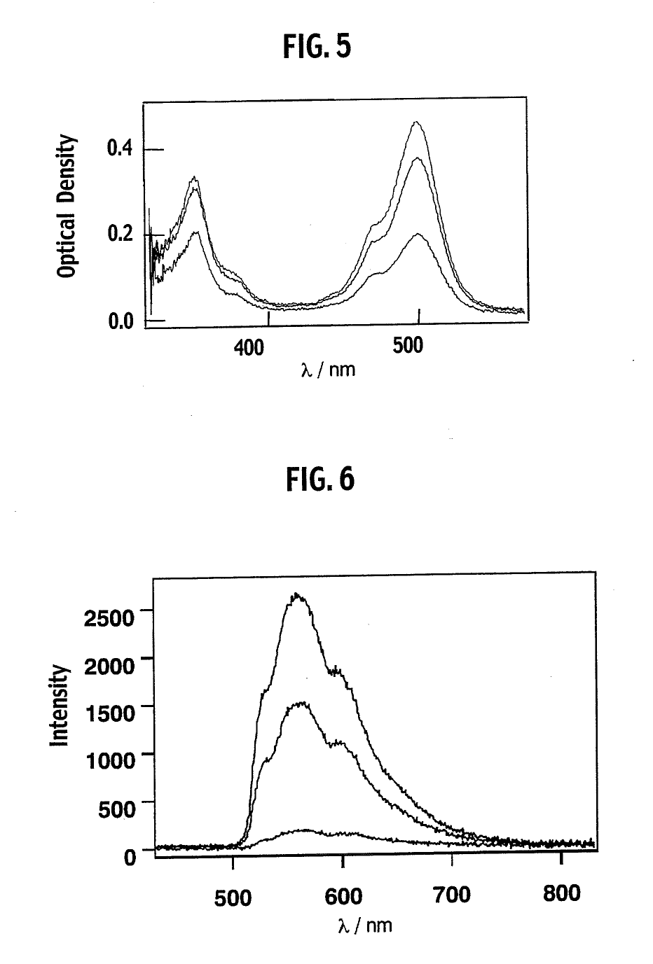

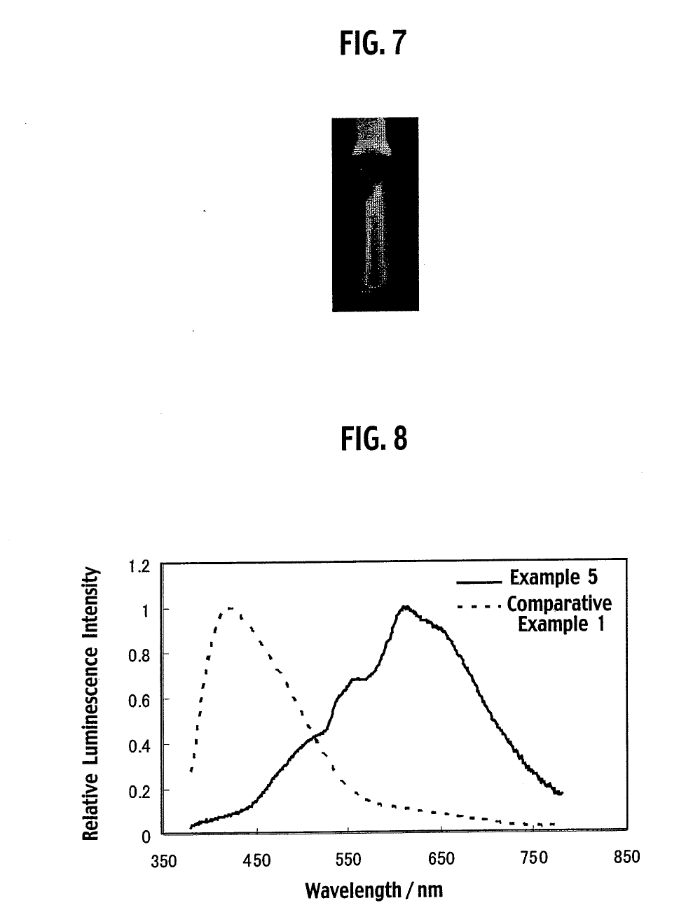

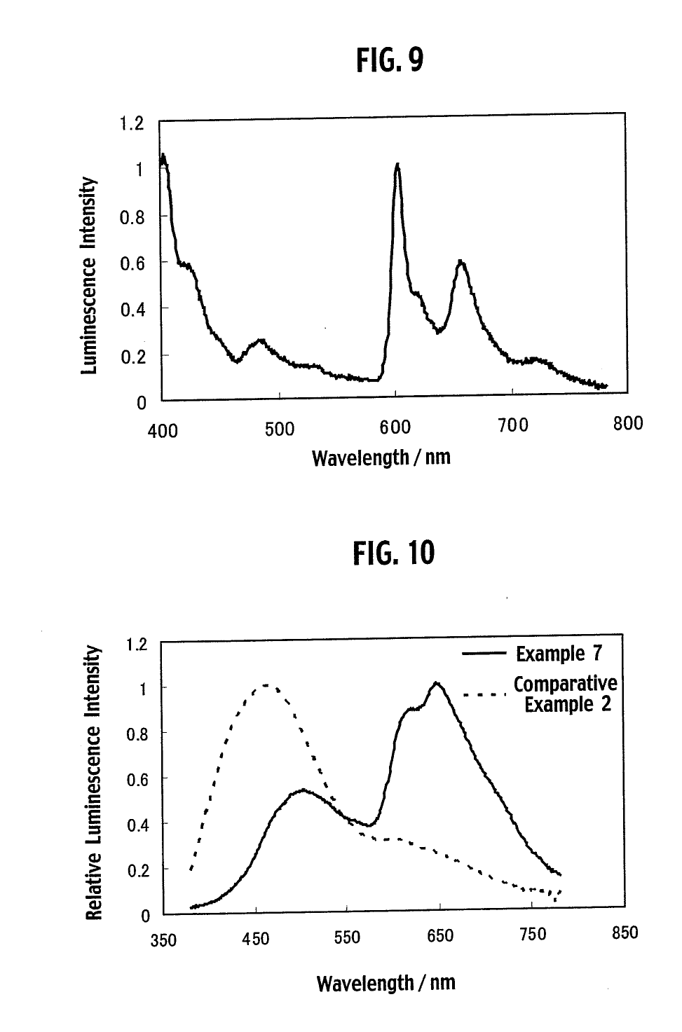

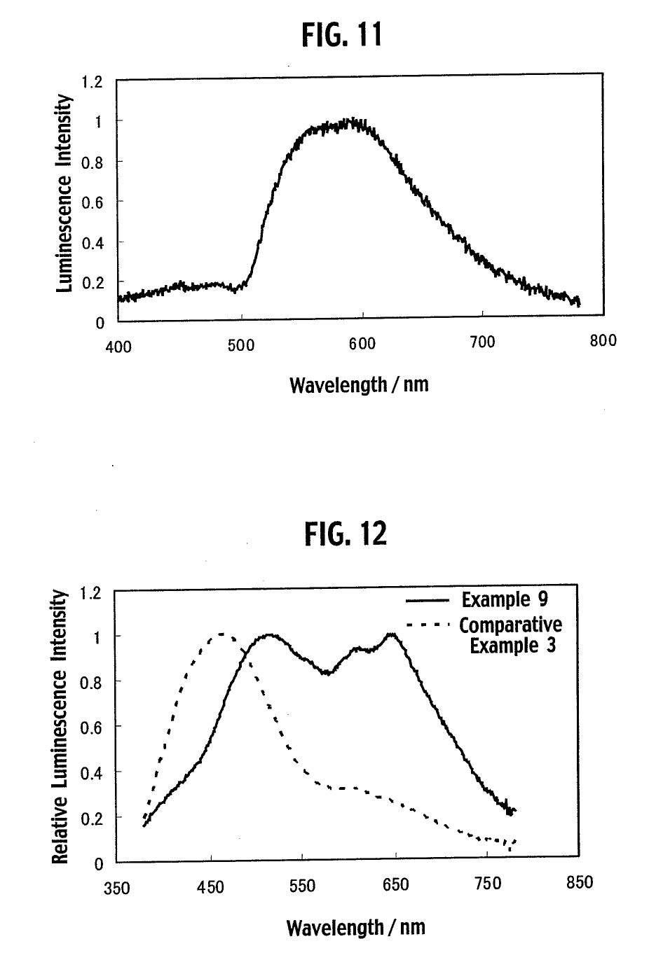

1. A method of luminescence of a chemical substance, comprising:
injecting an electric charge into the chemical substance so as to form an oxidized form or a reduced form of another chemical substance having a chemical structure that is different from that of the chemical substance, and
further injecting an electric charge that is opposite to the above electric charge so as to form an excited state of the another chemical substance to thereby make it luminesce.

2. The method of luminescence according to claim 1, wherein the another chemical substance turns back into the chemical substance after luminescence.

3. The method of luminescence according to claim 1, wherein the another chemical substance is formed via a bond formation reaction from the chemical substance.

4. The method of luminescence according to claim 1, wherein the another chemical substance is formed via a bond cleavage reaction from the chemical substance.

5. The method of luminescence according to claim 3, wherein the another chemical substance turns back into the chemical substance via a bond cleavage reaction.

6. The method of luminescence according to claim 4, wherein the another chemical substance turns back into the chemical substance via a bond formation reaction.

7. The method of luminescence according to claim 1, wherein the another chemical substance is an open-shell species having a monoradical or biradical.

8. The method of luminescence according to claim 1, wherein the ground-state multiplicity of the another chemical substance is a triplet.

9. The method of luminescence according to claim 1, wherein the chemical substance is represented by Formula (1) below: 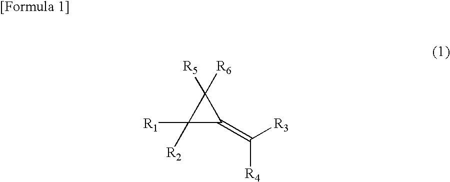
(in the formula, R₁ to R₆ denote a hydrogen atom, a halogen atom, a cyano group, a nitro group, a hydroxyl group, or a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, R₁ to R₆ may be identical to or different from each other; and, furthermore, R₁ to R₆ may have a substituent selected from the group consisting of —R₇, —OR₈, —SR₉, —OCOR₁₀, —COOR₁₁, —SiR₁₂R₁₃R₁₄, and —NR₁₅R₁₆ (here, R₇ to R₁₆ denote a hydrogen atom, a halogen atom, a cyano group, or a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₇ to R₁₆ may be identical to or different from each other)).

10. The method of luminescence according to claim 1, wherein the chemical substance is represented by Formula (4) below: 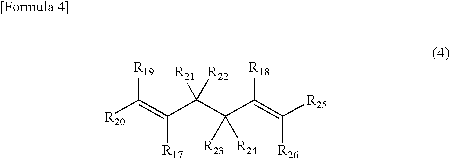
(in the formula, R₁₇ to R₂₆ denote a hydrogen atom, a halogen atom, a cyano group, a nitro group, a hydroxyl group, or a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, R₁₇ to R₂₆ may be identical to or different from each other; and, furthermore, R₁₇ to R₂₆ may have a substituent selected from the group consisting of —R₂₇, —OR₂₈, —SR₂₉, —OCOR₃₀, —COOR₃₁, —SiR₃₂R₃₃R₃₄, and —NR₃₅R₃₆ (here, R₂₇ to R₃₆ denote a hydrogen atom, a halogen atom, a cyano group, or a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₂₇ to R₃₆ may be identical to or different from each other)).

11. The method of luminescence according to claim 1, wherein the chemical substance is represented by Formula (7) below: 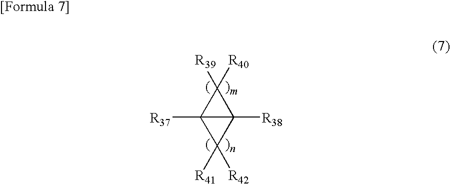
(in the formula, R₃₇ to R₄₂ denote a hydrogen atom, a halogen atom, a cyano group, a nitro group, a hydroxyl group, or a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, R₃₇ to R₄₂ may be identical to or different from each other; furthermore, R₃₇ to R₄₂ may have a substituent selected from the group consisting of —R₄₃, —OR₄₄, —SR₄₅, —OCOR₄₆, —COOR₄₇, —SiR₄₈R₄₉R₅₀, and —NR₅₁R₅₂ (here, R₄₃ to R₅₂ denote a hydrogen atom, a halogen atom, a cyano group, or a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₄₃ to R₅₂ may be identical to or different from each other), and m and n are integers of 1 to 3).

12. The method of luminescence according to claim 1, wherein the chemical substance is represented by Formula (10) below: 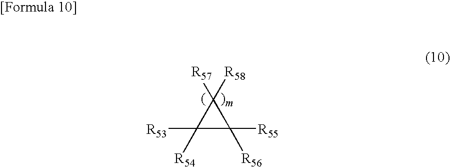
(in the formula, R₅₃ to R₅₈ denote a hydrogen atom, a halogen atom, a cyano group, a nitro group, a hydroxyl group, or a mercapto group; a straight-chain, cyclic, or branched alkyl group, alkoxy group, or alkylthio group having 1 to 22 carbons; an aryl group having 6 to 30 carbons, a heteroaryl group having 2 to 30 carbons, an aryloxy group having 6 to 30 carbons, a heteroaryloxy group having 2 to 30 carbons, an arylthio group having 6 to 30 carbons, a heteroarylthio group having 2 to 30 carbons, or an aralkyl group having 7 to 30 carbons, R₅₃ to R₅₈ may be identical to or different from each other; furthermore, R₅₃ to R₅₈ may have a substituent selected from the group consisting of —R₅₉, —OR₆₀, —SR₆₁, —OCOR₆₂, —COOR₆₃, —SiR₆₄R₆₅R₆₆, and —NR₆₇R₆₈ (here, R₅₉ to R₆₈ denote a hydrogen atom, a halogen atom, a cyano group, or a nitro group; a straight-chain, cyclic, or branched alkyl group having 1 to 22 carbons, or a halogen-substituted alkyl group in which part or all of the hydrogen atoms of the above are substituted with a halogen atom; an aryl group having 6 to 30 carbons, a heteroaryl group having 2 to 30 carbons, or an aralkyl group having 7 to 30 carbons, or a halogen-substituted aryl group, halogen-substituted heteroaryl group, or halogen-substituted aralkyl group in which part or all of the hydrogen atoms of the above are substituted with a halogen atom, and R₅₉ to R₆₈ may be identical to or different from each other), and m is an integer of 1 to 3).

13. A luminescent device comprising a chemical substance for luminescence, wherein the chemical substance changes into another chemical substance having a chemical structure that is different from that of the chemical substance and that the another chemical substance luminesces.

14. An electroluminescent device comprising a chemical substance for luminescence, wherein the chemical substance changes into another chemical substance having a chemical structure that is different from that of the chemical substance and that the another chemical substance luminesces.
